(19) United States
(12) Plant Patent Application Publication
(10) Pub. No.: US 2015/0163977 P1
(43) Pub. Date: Jun. 11, 2015

(54) Paulownia variety denominated Belle Select #2

(50) ;

(71) Applicant: Dennis Beeson, Bakersfield, CA (US)

(72) Inventor: Dennis Beeson, Bakersfield, CA (US)

(21) Appl. No.: 13/998,809

(22) Filed: Dec. 9, 2013

(51) Int. Cl. A01H 5/00

(52) U.S. Cl. PLT/216

(57) ABSTRACT

The invention relates to a new and distinct variety of Paulownia tree denominated “Belle.” The invention is a rapidly-growing variety distinguished from its parent varieties in terms of flower color, time of bloom, seed pod size and shape, and rate of growth.

### RELATED APPLICATIONS

Not applicable.

### BACKGROUND OF THE INVENTION

1. Field of the Invention

The present invention relates to a tree of the genus Paulownia.

2. Background

The Paulownia genus includes from nine to twelve species, depending on the taxonomic authority consulted. The plants are in the family Paulowniaceae, and are related to, and sometimes included in, the family Scrophulariaceae, commonly known as the Figwort family. The Paulownia tree is a deciduous tree native to China and known for its rapid growth, profuse spring bloom of foxglove-like flowers, and large catalpa-like green leaves.

Paulownia is important for lumber production, owing largely to its rapid growth. The wood of the tree is also used in the production of chests, boxes, and some musical instruments, as well as any other use for which a fine-grained, soft wood is desirable.

### SUMMARY OF THE INVENTION

The present invention relates to a new and distinct variety of Paulownia tree. The new cultivar, denominated “Belle Select #2,” (hereinafter referred to as “Belle”), was parented via the uncontrolled pollination of Paulownia fortunei and a rapid-growth genetic hybrid Paulownia tree referred to by the inventor as “Megafolia.” Megafolia is a cross between a Fortunei Select #2 clone referred to by the inventor as “Baby Huey,” and a Paulownia kawakamii. Baby Huey was discovered by the inventor as a chance seedling on his Paulownia tree farm in Bakersfield, Calif. Megafolia is believed to have been parented from the Paulownia tree known as Baby Huey based on its characteristics and close proximity to Baby Huey in the area of discovery. Baby Huey was known by the inventor to be a Paulownia fortunei Select #2 clone, and was selected by the inventor due to its rapid seed emergence and subsequent remarkable rate of growth.

The Belle cultivar was selected by the inventor due to its relative vigor and rapid growth as compared to other Paulownia seedlings, its early bloom emergence, the relatively small size of its seed pods, and the deep purple color of its flowers. These characteristics serve to readily distinguish Belle from its parent plants. Belle has flowers of a deeper purple color than Paulownia kawakamii, an approximately four to five week earlier bloom, smaller rounder seed pods, and an approximate 20% increase in growth rate. Belle is distinguished from Paulownia fortunei in having deep purple rather than white lavender blooms, an approximately three week earlier bloom, much smaller rounder seed pods, and an approximate 10% increase in rate of growth.

The propagated trees of Belle have been determined to be stable, reproducible, and true to type in successive generations, with each generation exhibiting consistently higher growth rates than any other Paulownia species known to the inventor.

### BRIEF DESCRIPTION OF THE DRAWINGS

FIG. 1 is a color photograph showing the typical size, shape, and color of the blossoms of the new variety in summer, in Bakersfield, Calif.

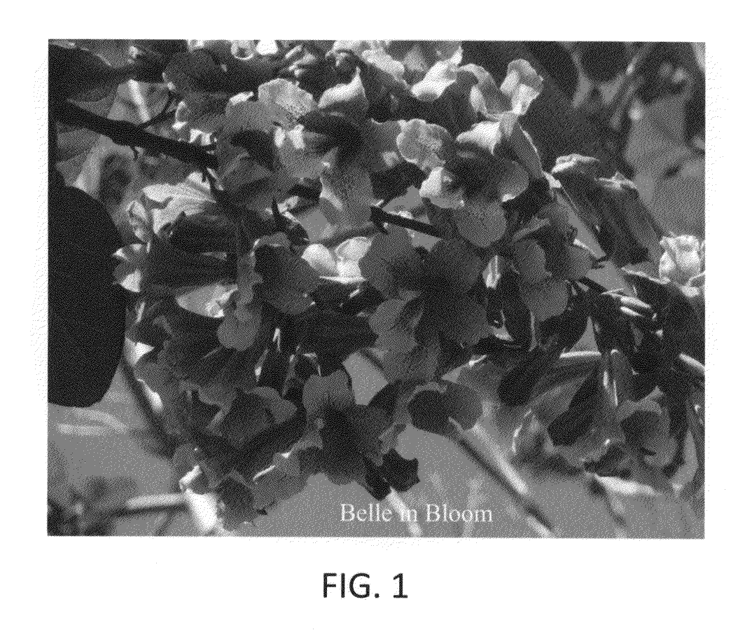

FIG. 2 is a color photograph of the new variety of FIG. 1 showing the typical size, shape and color of seed pods of the new variety, in contrast to see pods from the variety referred to herein as Megafolia.

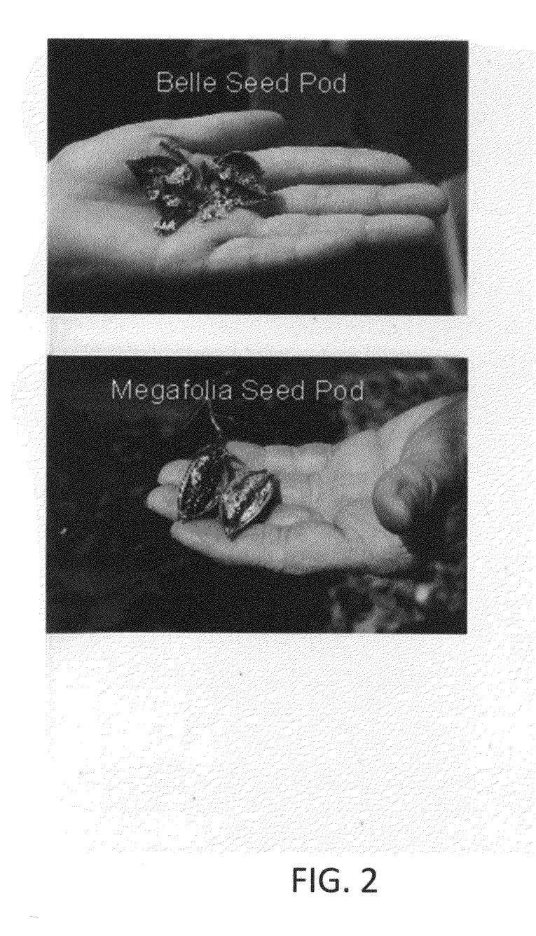

FIG. 3 is a color photograph of the new variety of FIG. 1 showing the typical coloration of growth found on the inner part of the current variety during summer, with the color ranging from green foliage to leaves with advancing dark green from the leaf petiole and the leaf base down the leaf blade.

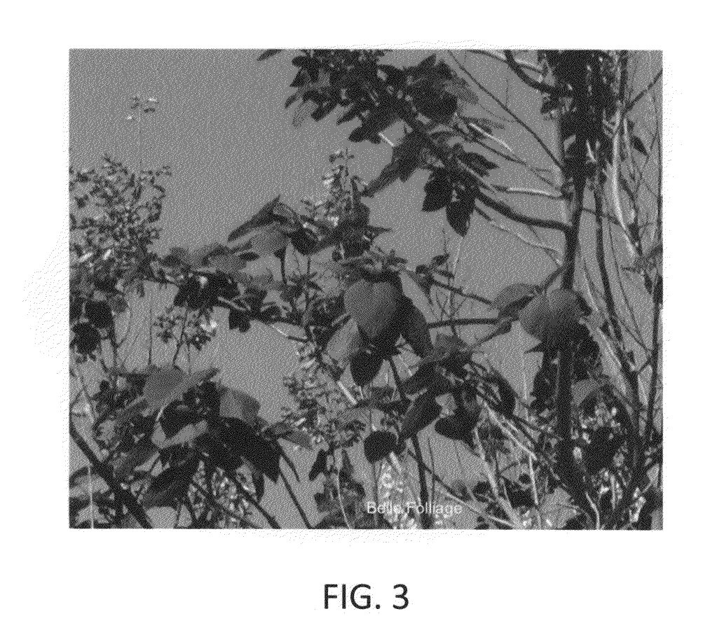

FIG. 4 is a color photograph showing a close view of a flower of the new variety of FIG. 1.

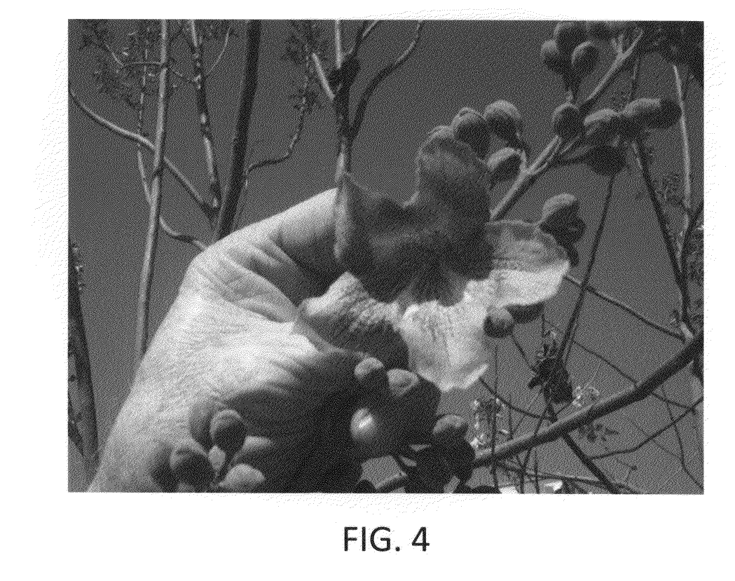

### DETAILED BOTANICAL DESCRIPTION

The new and distinct variety of Paulownia described herein has a number of stable, representative characteristics that have been observed in representative specimens of the variety, grown and observed in Bakersfield, Calif. The Belle cultivars grown and observed are distinguished from other Paulownia varieties by a set of characteristics exhibited by the new variety. Belle cultivars typically grow to a height of over twenty feet within one year, and are suitable for harvesting for commercial lumber within three years. The new variety also exhibits limbs that are upright to spreading, with a rounded crown. Belle cultivars have ovate, green leaves, up to 5″ to 7″ in length, that are lightly hairy above and densely hairy and sticky beneath the leaves. The variety also have a profuse bloom of fragrant, tubular, funnel-shaped purple flowers, up to 2½″ in length, with interior dark purple spotting and creamy yellow striping. The flowers appear in spring, in clusters up to 14″ long, before the appearance of the foliage. The aroma of the flowers is reminiscent of lilac. The flowers are followed by oval, woody, dehiscent seed capsules that emerge sticky green and ripen to brown in fall, at which point they split open, releasing abundant seeds. Belle exhibits tree bark that is gray in color and flaky in texture. The sap of the tree is very sweet in flavor and, when measured by the Brix test using a refractometer, is comparable to a pineapple (i.e. from 24+ on the Brix scale).

The following is a botanical description of the characteristics of the new variety as displayed by a typical two year-old specimen grown in Bakersfield, Calif. References to color are made in accordance with the colors on the R.H.S. Colour Chart (2005 ed.), published by The Royal Horticultural Society, London, England. Coloration of leaves and bark may be variable due to conditions of nutrition, stress, age of tree, location on tree, and the presence or absence of sun or shade. Tree part comparisons have been made from a mature tree, where growth rates and characteristics are considered typical.
Flowers: Inconspicuous purplish lavender true flowers in sessile in compact umbels are surrounded by a showy white involucre much exceeding the flowers. The true flowers are (Dark Blue Violet 93A) at petal tip and (Yellow-Green Group 15D) at petal base on the lower surfaces The rounded, central umbel is approximately 1 cm wide by 1 cm high at the top of a peduncle 5 to 6 cm in length and White 155C. The valvate — 4 petaled flowers consist of four stamens (White 155C) with filament (Dark Violet 83A) of 2 mm length by 0.5 mm wide. The stigma is flat, maturing to (Dark Violet 83A) and 0.5 mm in width. The number of inconspicuous flowers will vary per umbel and virtually open at the same time — opening from outside in. The involucre of four partially overlapping smooth, entire, oval-acuminate creamy white bracts form a four-pointed symmetrical star characterizing each individual inflorescence. Initially, bracts with rounded base and acuminate apices are white (White 155C) on upper side and (White 155C) underneath. Mature bracts resemble (White 155C) above, (White 155A), below. Typical and observed bract size averages 4.5 to 5 cm long and 3.5 to 4 cm wide, thereby producing an inflorescence with an overall width of 9 to 10.5 cm. Flowering commences the last week of April to the first week of May, in Bakersfield, Calif., lasting about three to four weeks. Flowers of this cultivar are borne more on the inside of the tree and are not abundant.
Fruits: The fruits connate into a globular, fleshy head, predominantly green (Dark Green136A) and approximately 3 cm in diameter at maturity. The syncarp is initially held upright on a 2 to 3 cm peduncle, later becoming pendulous.
Buds: Buds are two types — Globose, tapering flower buds averaging 7 mm in length by 5 mm in width at the base, color green (Dark Green 137A) and sharply tapered vegetative buds averaging 4 mm in length by 2 mm in width at base, color green (Green 137C).
Leaves: Deciduous, simple, entire leaf, elliptic ovate with acuminate tips and cuneate bases, 5 to 12 cm long and 3.5 to 7.5 cm wide. Leaf petioles are 5 to 10 mm long by 2 mm in diameter on average and of the color green (Green 144A). Leaf blades are glabrous on top, glabrous underneath, and lacking tufts of hairs in leaf vein axils on the underside of leaf. Newly emerging leaves in the spring exhibit green (Green 137A) on the upper side of the leaves and (Green 144C/144A) underneath, darkening to green (DarkGreen 137A) on the upper side of leaves and (Green Group 139B) below. Many leaves of the current year's growth flush became yellow (Yellow Group 138) by late summer. The yellow coloration is exhibited from leaf petiole and base down leaf blade and may include from ⅓ to entire leaf. Coloration begins in September. Overall, the current season's growth found on the inner part of the tree may range from green foliage to leaves with yellow advancing down the leaves to varying degrees. The more vigorous new growth on the outside canopy exhibits mainly solid green leaves. At maturity, yellow leaves develop orange blushes (Orange Brown N170A) with the orange predominating. Much later, these blushed yellow-orange leaves become infused with more red (Brown Orange 171B). Leaves that have remained green will later exhibit red (Red Group 46B and 46C) and orange (Red Group 42B and 42C) fall colors typical of the species. The yellow coloration of the foliage appears year after year regardless of weather conditions.
Stems: Young stems are initially slender, Dark Green (Green 137A). Typical observed lenticel height is 1 mm times 0.5 mm diameter. 45 to 68 lenticels are present per square centimeter.
Branches: New shoots are green (Dark Green 141A turning to dark green (Dark Green 137A) over time. Mature branch color is green (Dark Green 144A).
Form, size and branching characteristics: A shrubby tree six to ten meters in height and spread at maturity. Rounded as a young tree, maintaining that shape with a layered, horizontal branching habit. A wide range of factors, especially age, location, and whether the tree is multi-stem or single stem, influences the size of stems and branches. There is no predictable correlation between the diameter of a primary stem and any branch arising from it.
Growth rate: Rapid, more robust in youth.

Having thus described the preferred embodiment of the invention, what is claimed as new and desired to be protected by Letters Patent includes the following:

1. A new and distinct variety of Paulownia tree as substantially illustrated and described herein.
